(19) United States
(12) Patent Application Publication
(10) Pub. No.: US 2022/0044395 A1
(43) Pub. Date: Feb. 10, 2022

(54) SYSTEM, METHOD AND INTERFACE FOR COMMUNICATION AND VIEWING OF MEDICAL IMAGING

(71) Applicant: INTEGRAL SYSTEMS PTY LTD, Hamilton, NSW (AU)

(72) Inventors: Houman EBRAHIMI, Pymble, NSW (AU); Kenny Zhihong HONG, Pymble, NSW (AU); Steven Paul NICKLIN, Pymble, NSW (AU)

(21) Appl. No.: 17/276,457

(22) PCT Filed: Sep. 13, 2019
(86) PCT No.: PCT/AU2019/050987

§ 371 Date: Mar. 15, 2021

(30) Foreign Application Priority Data
Sep. 16, 2018 (AU) 2018903484

(51) Int. Cl. G06T 7/00; G16H 30/20; G06F 16/58; G06T 9/00

(52) U.S. Cl. CPC G06T 7/0012; G06T 9/00; G06F 16/58; G16H 30/20

(57) ABSTRACT

A system for processing, communication and viewing of medical imaging study data is configured to process and communicate medical imaging study data for communication between a local system and a client terminal. The systems preferably include a local system, one or more client terminals and an intermediate remote server. The local system may be located within a Virtual Private Network (VPN) and includes a local or site server, a Picture Archiving and Communication System (PACS) and a Radiology Information System (RIS). The local or site server communicates between the PACS and the RIS, and the intermediate remote server that may be outside of the VPN. The local or site server may be configured to perform a lossless image processing method to process, compress and encrypt and communicate medical imaging study data. An interface and associated methods of viewing medical imaging study data are also disclosed.

### RELATED APPLICATIONS

This application claims priority from Australian provisional patent application no. 2018903484 filed on 16 Sep. 2018, the contents of which are incorporated by reference.

### TECHNICAL FIELD

The invention relates to a system, a method and an interface for medical communication and viewing of imaging. In particular, the invention relates to a system, a method and an interface for radiology images in relation to teleradiology services.

### BACKGROUND

Teleradiology involves the transmission of radiology patient images, such as X-rays, CT scans, and MRIs, from one location to another for the purposes of remote report creation and sharing studies with other radiologists and physicians.

Existing systems common in radiology include the Picture Archiving and Communication System (PACS) that manages the imaging data for a case, and the Radiology Information System (RIS) that manages documents, patient information and case information.

Such systems are often located within a Local Area Network (LAN) at the site and are required to be access by a VPN (Virtual Private Network). A problem with such systems is that multiple software applications may be required to access the services at the site via the VPN. This may result in the initial setup being complex, and in some cases require dedicated hardware. Further, the VPN performance may be limited by the upload bandwidth of the imaging site, which may be inadequate.

Medical imaging data are often large files that comprise sensitive information. Medical images used for reporting must also be compressed in a manner such that no information is lost, referred to as “lossless” compression. Accordingly, if such images are to be transferred by a network, a problem exists in relation to how to secure the files and also how to reduce the file size to improve file transfer speed. A problem also exists with ensuring compression and decompression of the images is lossless.

Medical images, when received by a physician, are required to be viewed across a sequence of different projections, cross-section planes, and reconstruction algorithms. Often, a physician will wish to view a particular sequence of cross-sections and projections to better assist the diagnosis process. Accordingly, a problem exists with viewing such images, in particular, when multiple views are desired to be simultaneously viewed and customised to the requirements of a particular case or physician.

The invention disclosed herein seeks to provide an improved teleradiology system, methods of image compression and communication, and an interface for viewing medical imaging data, or at least provide useful alternatives.

### SUMMARY

In accordance with a first broad aspect there is provided, a method for communicating medical imaging study data between a local system and a client terminal, the method including one or more of the following steps: processing, at the local system, the medical imaging study data by separating the study data into image data and associated meta data; compressing, at the local system, the image data to provide compressed image data; creating, at the local system, a first file type including the compressed image data and associated meta data; and at the local system, at least one of encrypting and further compressing the first file type suitable for communication with the client terminal.

In accordance with a second broad aspect there is provided, a method for processing medical imaging study data for communication between a local system and a client terminal, the method including the steps of: processing, at the local system, the study data into image data and associated meta data; compressing, at the local system, the image data; creating, at the local system, a first file type including the compressed image data and associated meta data; processing, at the local system, the compressed image data associated with a plurality of the first file types to determine compatible compressed images based on a compatibility criteria; creating, at the local system, a second file type by combining a plurality of the compatible compressed images into a video format; at the local system, at least one of encrypting and further compressing one or more of the first file type and the second file type suitable for communication with the client terminal.

In accordance with a third broad aspect there is provided, a method for communication of medical imaging study data between a local system and a client terminal, the method including the steps of: processing, at the local system, the study data into image data and associated meta data; compressing, at the local system, the image data; creating, at the local system, a first file type including the compressed image data and associated meta data; processing, at the local system, the compressed image data associated with a plurality of the first file types to determine compatible compressed images based on a compatibility criteria; creating, at the local system, a second file type by combining a plurality of the compatible compressed images into a video format; at the local system, at least one of encrypting and further compressing one or more of the first file type and the second file type to provide transferable file data suitable for communication with the client terminal; communicating, via a remote server in communication between the local system and the remote system, the transferable file data to the client terminal; and at the client terminal, at least one of decrypting and decompressing, the transferable file data to extract the one or more of the first file type and the second file types; and processing, at the client terminal at least the second file type to create a plurality of the first file types from each of the second file type.

In an aspect, the step of creating the second file type further includes: creating a mapping file to link video frames of the video format to the associated metadata files.

In another aspect, the compatibility criteria includes: image dimensions of the compressed image data must be equal; and an instance of medical imaging study data of the must contain a single frame.

In yet another aspect, the image data is compressed to using as lossless compression method, such as PNG, to provide the compressed image data, and wherein the plurality of the compatible compressed images are combined with a lossless video compression method, such as FFmpeg, to provide the video format.

In yet another aspect, the local system is within a virtual private network.

In accordance with a fourth broad aspect there is provided, a system for teleradiology, the system including a local system and a remote client terminal, the system being configurable to: process, at the local system, medical imaging study data by separating the study data into image data and associated meta data; compress, at the local system, the image data; create, at the local system, a first file type including the compressed image data and associated meta data, a plurality of the first file types being creatable for a plurality of instance data of the study data; and at the local system, at least one of encrypting and further compressing the first file type suitable for communication with the client terminal.

In accordance with a fifth broad aspect there is provided, a system for processing medical imaging study data for communication between a local system and a client terminal, the system including a local system and a remote client terminal, the system being configurable to: process, at the local system, the study data to into separate image data and associated meta data; compress, at the local system, the image data; create, at the local system, a first file type including the compressed image data and associated meta data; process, at the local system, the compressed image data associated with a plurality of the first file types to determine compatible compressed images based on a compatibility criteria; create, at the local system, a second file type by combining a plurality of the compatible compressed images into a video format; at the local system, at least one of encrypt and further compress one or more of the first file type and the second file type suitable for communication with the client terminal.

In accordance with a sixth broad aspect there is provided, a system for communication of medical imaging study data between a local system and a client terminal via an intermediate remote server, the system being configurable to: process, at the local system, the study data into image data and associated meta data; compress, at the local system, the image data; create, at the local system, a first file type including the compressed image data and associated meta data; process, at the local system, the compressed image data associated with a plurality of the first file types to determine compatible compressed images based on a compatibility criteria; create, at the local system, a second file type by combining a plurality of the compatible compressed images into a video format; at the local system, at least one of encrypt and further compress one or more of the first file type and the second file type in transferable file data suitable for communication with the client terminal; communicate, via the remote server, the transferable file data to the client terminal; and at the client terminal, at least one of decrypt and decompress, the transferable file data to extract the one or more of the first file type and the second file types; and process, at the client terminal, at least the second file type to convert the video format into a plurality of the first file types from each of the second file type, each of the first file types including the compressed image data and associated metadata.

In accordance with a seventh broad aspect there is provided, a server for processing and communication of medical imaging data, the server being configurable to: receive study data of the medical imaging data from one or more local systems; process the study data to separate the study data into image data and associated meta data; compress the image data to form compressed image data; create a first file type including the compressed image data and associated meta data; process the compressed image data associated with a plurality of the first file types to determine compatible compressed images based on a compatibility criteria; create a second file type by combining a plurality of the compatible compressed images into a video format; at least one of encrypt and further compress one or more the first file type and the second file type into transferable file data suitable for communication with the client terminal; and communicate the transferable file data to a client terminal.

In accordance with an eighth broad aspect there is provided, a teleradiology system for functioning with a Picture Archiving and Communication System (PACS) and the Radiology Information System (RIS), the system including a local server in proximate communication with the PACS and RIS, the local server being configurable to undertake one or more of the following: receive Digital Imaging and Communications in Medicine (DICOM) files from the PACS; retrieve prior imaging data from PACS; at least one of compress and encrypt of data from DICOM files to form transferable compressed data; upload the transferable compressed data to a cloud server; retrieve of one or more prior reports from the RIS; upload of one or more prior reports to the cloud server; retrieve one or more request documents from RIS; upload of the one or more request documents to the cloud server; retrieve one or more validated reports from the cloud server; and communicate the one or more validated report to the RIS.

In accordance with a ninth broad aspect there is provided a configurable interface for display of medical image data, the interface being shown on a window of a computer display, the interface including: a draggable icon displayable on the window that is selectable to show an image of the medical image data, the draggable icon including a thumbnail image representative of the image and an indicia to indicate associated image information; and a drop zone on the window, the drop zone being adapted to indicate a location of the window when the draggable icon is dragged thereover in which the image will be displayable.

In an aspect, the draggable icon is at least one of a series icon that represents a single imaging series and a viewer icon representing a viewer displaying an imaging series.

In another aspect, the interface includes a series viewer, the series view being a portion of the window that displays imaging from a single series.

In yet another aspect, a plurality of drop zones are provided toward and around an edge of the window.

In yet another aspect, the interface includes a plurality of further drop zone locatable over the portion of the series viewer such that dragging at least one of the series icon and the viewer icon thereover enables the series viewer to be at least one of split, replaced, and swapped.

In accordance with tenth broad aspect there is provide, a method of configuration of an interface of a window for viewing medical image data, the method including the steps of: providing a draggable icon displayable on the window that is selectable to show an image of the medical image data, the draggable icon including a thumbnail image representative of the image and an indicia to indicate associated image information; and providing a drop zone on the window, the drop zone being adapted to indicate a location of the window when the draggable icon is dragged thereover in which the image will be displayable.

### BRIEF DESCRIPTION OF THE FIGURES

The invention is described, by way of non-limiting example only, by reference to the accompanying figures, in which;

FIG. 1a is a system diagram illustrating an example teleradiology system including a local or site system, a remote or cloud system and one or more client terminals;

FIGS. 1b and 1c are flow diagrams that illustrate methods of use of the teleradiology system illustrated in FIG. 1;

FIG. 2a is a functional diagram illustrating the teleradiology system and, in particular, the local server and data handling operations thereof;

FIG. 2b flow diagram illustrating a method of use of the teleradiology system, and in particular the local server and data handling operations thereof, as illustrated in FIG. 2a;

FIG. 3 is a system diagram illustrating a microservices architecture of the cloud system;

FIG. 4 is a flow diagram illustrating a method for compression of medical image data for communication within the teleradiology system;

FIG. 5 illustrates a file format of the first file type for a single frame instance;

FIG. 6 illustrates a file format of the first file type for a multi-frame instance;

FIG. 7 illustrates a file format of the second file type;

FIG. 8a illustrates an example screen of a GUI having an example set of views and a view selection bar or region having series and viewer icons;

FIG. 8b illustrates an example screen of a series viewer;

FIGS. 9a and 9b respectively illustrate an unselected and a selected series icon;

FIG. 10 illustrates a viewer icon;

FIG. 11 illustrates window drag and drop zones to add a new series viewer;

FIG. 12 illustrates the add series viewer drop indicator on the left of the window;

FIG. 13 illustrates split/replace viewer drop zones within the series viewer;

FIG. 14 illustrates the split drop indicator within the top left viewer;

FIG. 15 illustrates the replace/swap viewer drop indicator within the top left viewer;

FIG. 16 illustrates in overlay preview displaying the series from the left in blue over the existing series on the right; and

FIGS. 17a to 17c respectively illustrate closing series viewer C or D, closing series viewer E, and closing series viewer A or B.

### DETAILED DESCRIPTION

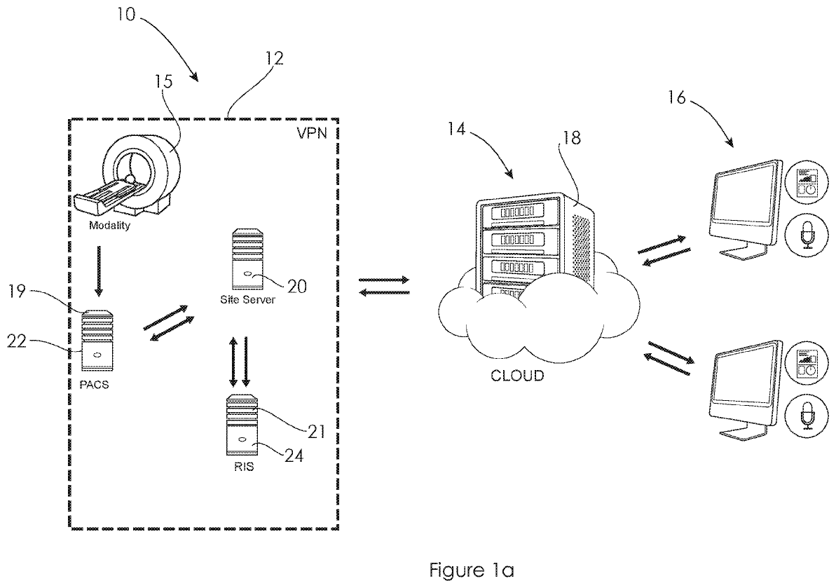

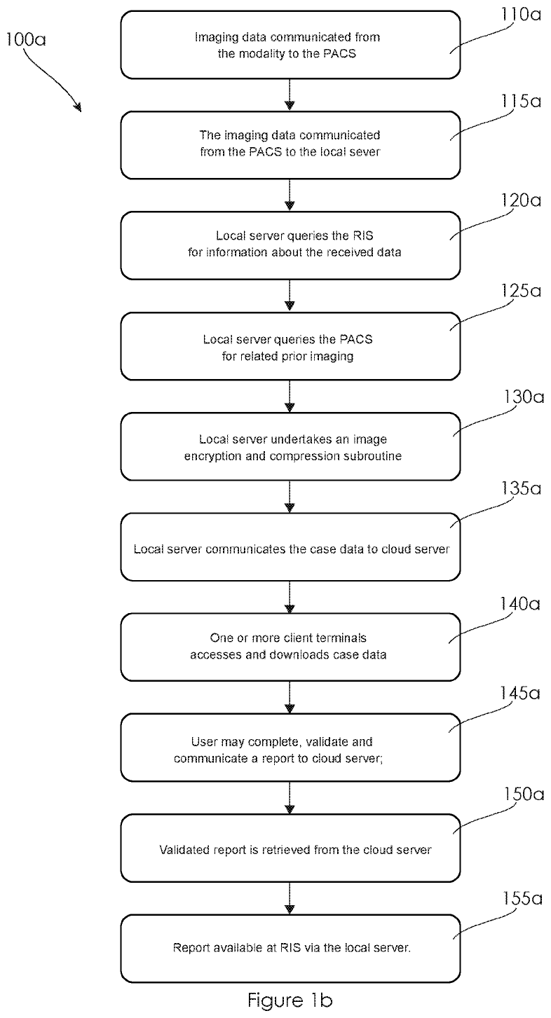

Referring to FIG. 1 there is shown a system 10 for teleradiology. The system 10 including a local or site system 12, a remote or cloud system 14 and one or more client terminals 16. The local system 12 is typically located at a site of collection of radiology information and medical imaging data such as a radiology practice, hospital or imaging centre, the remote system 14 may be located remote to the site at any suitable location and the one or more client terminal 16 may be also be located at any suitable location, either onsite or remote.

The one or more client terminals 16 may be usable by a user (i.e. a physician) to view and make diagnoses based on the of radiology information and the remote system 14 may include one or more cloud servers 18 to serve and communicate information between the local system 12 and the one or more client terminals 16.

Turning firstly to the local system 12, the system 10 includes a local or site server 20 located generally near to the point of radiology image collection apparatus referred to as a modality 15. Such a modality 15 may include, but not limited to, X-ray, ultrasound, computed tomography (CT), nuclear medicine including positron emission tomography (PET), and magnetic resonance imaging (MRI).

The local server 20 is configured to interface with a first system 19 that may be, for example, a Picture Archiving and Communication System (PACS) 22 that handles imaging data and a second system 21 that may be, for example, a Radiology Information System (RIS) 24 that handles documents and patient data. In some examples, the first and second systems 19, 21 may take other forms and could be combined. It is noted that the local or site server 20 may include one or multiple computers with memory or storage, such as one functioning for the data compression and the other handling communication.

Typically, most radiology practices or imaging centres will already have existing PACS and RIS systems 22, 24 in place, and in this example the local server 20 is configured to interface with these systems within a Virtual Private Network (VPN). The PACS and RIS systems may include PACS and RIS computers or systems of computers, and associated storage or memory such as databases.

It is noted that various implementations of the local system 12 are possible, and the described implementation herein is for example purposes only. However, the local server 20 is advantageous as the local server 20 may enable and perform data operations on image data and study data within the VPN and any associated firewalls.

The local server 20 communicates with the one or more client terminals 16 via the cloud server 18 that provides a common point of contact or communication therebetween. The local server 20 thereby enabling communication between the local system 12 and external systems outside of the VPN and firewall.

A secure connection may be provided between the local server 20 and the remote server system 14 using SSL (Secure Sockets Layer) and/or TSL (Transport Security Layer). The remote system 14 including the cloud server 18 provides a cloud service including user accounts that makes user access and settings transferrable between locations. A suitable remote or cloud system 14 including the cloud server 18 is available from Amazon Web Services' (AWS), namely, the AWS On-demand cloud computing.

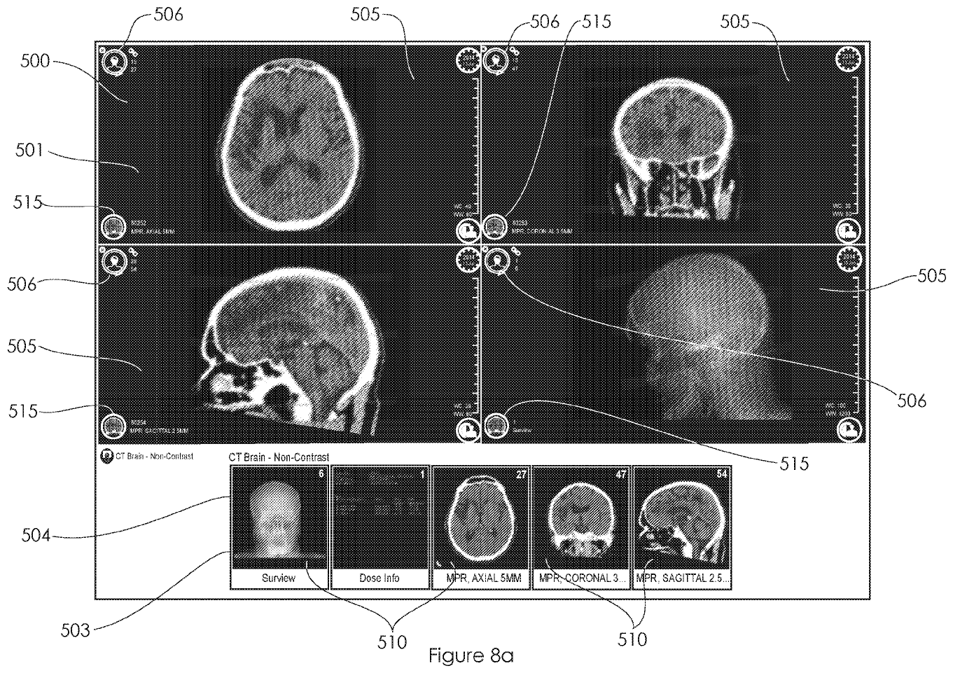

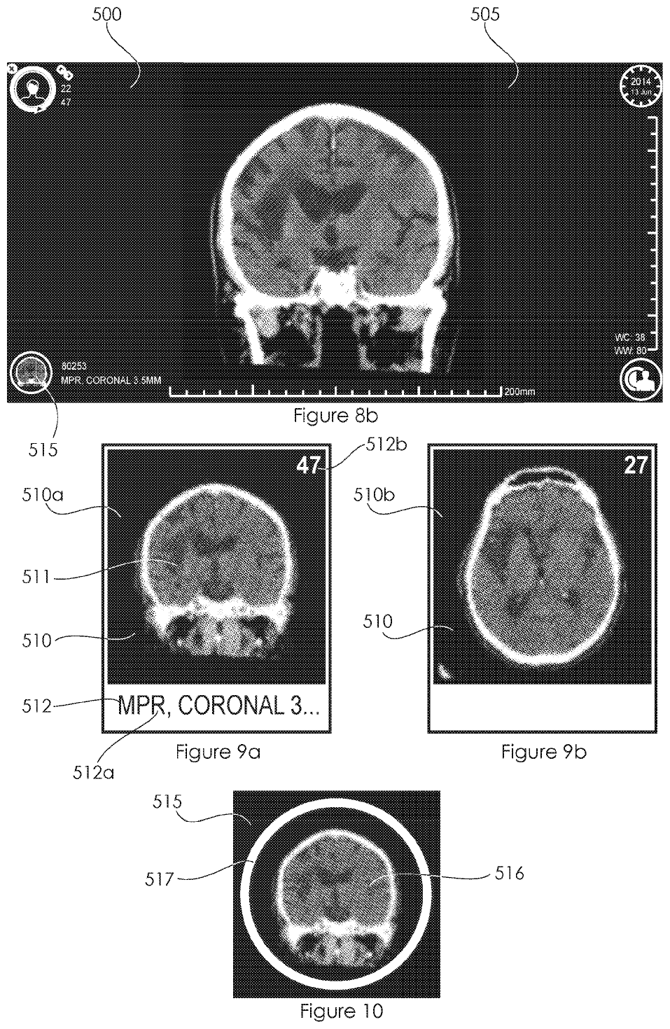

The one or more client terminals 16 may be any suitable computing or display device such as, but not limited to, personal computers, tablets, laptops and smart phones. Such computing devices may be Internet enabled for communication with the cloud server 18. Preferably, the one or more client terminals 16 are loaded with application software that is configured to send and receive data to the cloud server 18 and provide an interface 500, as shown for example in FIG. 8a, for viewing data received at the one or more client terminals 16, as is further detailed below in relation to FIGS. 8a to 17c. However, in other examples, the interface may be provided via an Internet browser with data operations occurring at the cloud server 18. Both examples are contemplated herein.

The system 10, in particular, the components thereof such as the cloud server 18 and the local server 20 are configured by application software to perform the method steps as set out herein.

Turning now to the operation of the system 10 in more detail, the system 10 may be configured to operate in one or more workflows or methods that may typically fall into one of two categories, PACS initiated or RIS initiated. Combinations of the PACS and RIS based workflows are possible, with RIS communication via either HL7 or proprietary API. An example of a PACS initiated method is 100a described below, followed by an example of a RIS initiated method 100b. Other methods are also possible and these are provided for example purposes only.

Referring to FIGS. 1a and 1b, the PACS initiated method 100a, may include at step 110a, sending or communicating imaging data collected from the modality 15 to the PACS system 22. The format of the images may initially be in a DICOM (Digital Imaging and Communications in Medicine) format. A DICOM data object consists of a number of attributes, including meta data items such as name, ID, etc., and also one special attribute containing the image pixel data.

In more general terms, the system data hierarchy may be categorised as follows:
A case may contain one or more imaging procedures;
An imaging procedure is classed as a study;
Each sequence of images within the study is classed as a series;
Each series contains one or more instances; and
Each instance contains one or more frames.

All of the above data may be broadly referred to as medical or radiology imaging data that includes a number of data subsets including images and meta data.

The method 100a may then further include, at step 115a, the imaging data being then communicated from the PACS 22 to the local site server 20. The imaging data may be moved using C-Move which is a well-known and defined operation of a DICOM system, and not described here in any further detail.

At step 120a, the local server 20 is configured to query the RIS 24 for information about the received data. This includes prior reports for that patient and request forms, and at step 125a the local server 20 is configured to query PACS 22 for related prior imaging. If related prior imaging is found, this imaging data is retrieved from the PACS 22 to be uploaded to the cloud server 18.

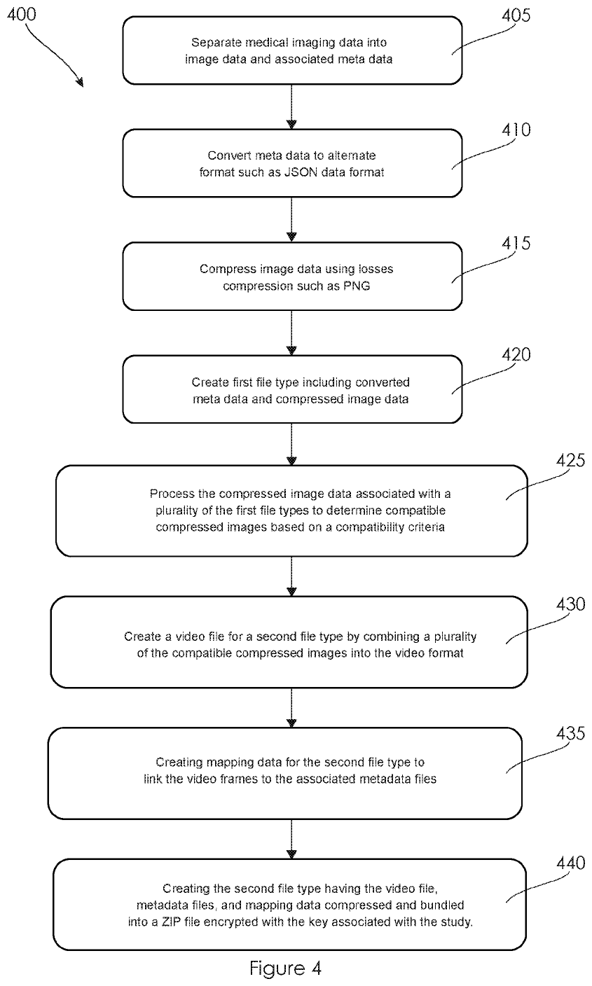

At step 130a, the local server 20 is configured to undertake an image encryption and compression subroutine 400, as detailed below with reference to FIG. 4, to create transferable case or study data communicable to the cloud server 18 and then to the one or more client terminals 16, as required. The case data may include one or more of study images, request documents, related prior patient image and related prior patient reports.

At the step 135a, the local server 20 is configured to upload the case data to the cloud server 18. The case data is storable at the cloud server 18. Again, the case data may include one or more of study images, request documents, related prior patient image and related prior patient reports.

At step 140a, the one or more client terminals 16 may access the case data and imaging data is downloaded to the relevant one or more client terminals 16 via the desktop application. These files may be pre-cached so that they are available before the user, that may be a radiologist, opens the case.

The radiologist may then view the case and image data. Preferably, the desktop application includes two components, a desktop server which runs in the background for precaching the study data, and the viewer application which provides the user interface for viewing imaging and documents and creating reports, as is further outlined below.

At step 145a, the radiologist may complete and validate a report. This report is then submitted and communicated to the cloud server 18. At step 150a, the validated report is retrieved from the cloud server 18 by the site server 20, and then available locally at site via the local server 20 and sent to the RIS 24, at step 155a.

Referring now to FIGS. 1a and 1c, a RIS initiated method 100b is briefly described. The RIS initiated method 100b share many similarities with the PACS initiated method 110b and as such each common step is not again described in described.

At step 110b, the local server 20 receives a HL7 message for a case to be reported. At step 115b, the local server receives HL7 message for prior reports. At step 120b the imaging data communicated from the PACS to the local sever 20 and at step 130b the local server 20 undertakes the image encryption and compression subroutine 400. At step 135b the local server 20 communicates the case data to cloud server and at step 140b the one or more client terminals 16 may accesses and download the case data. At step 145b, the user may complete, validate and communicate a report to cloud server 18. At step 150b, the validated report is retrieved from the cloud server 18, and at step 155b at the report is available at RIS via the local server 20.

Advantageously, the configuration of the system 10, in particular, the local server 20, enables communication with the cloud server 18 without using the VPN which simplifies configuration, allows greater bandwidth and hence speed and performance for the client terminals. The pre-caching (or pre-downloading) at the one or more client terminal avoids problems with streaming and improves speed and usability at the one or more client terminals 16 via the desktop application.

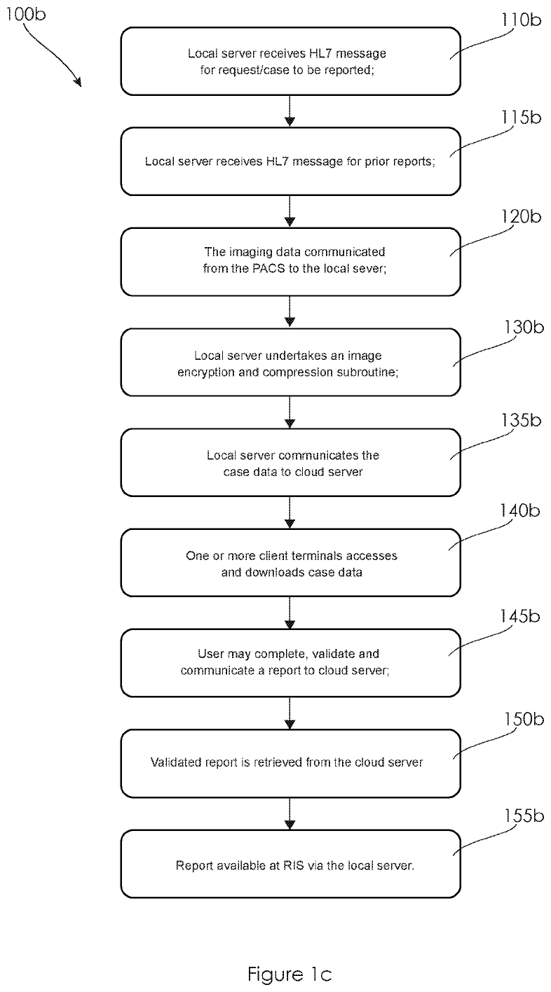

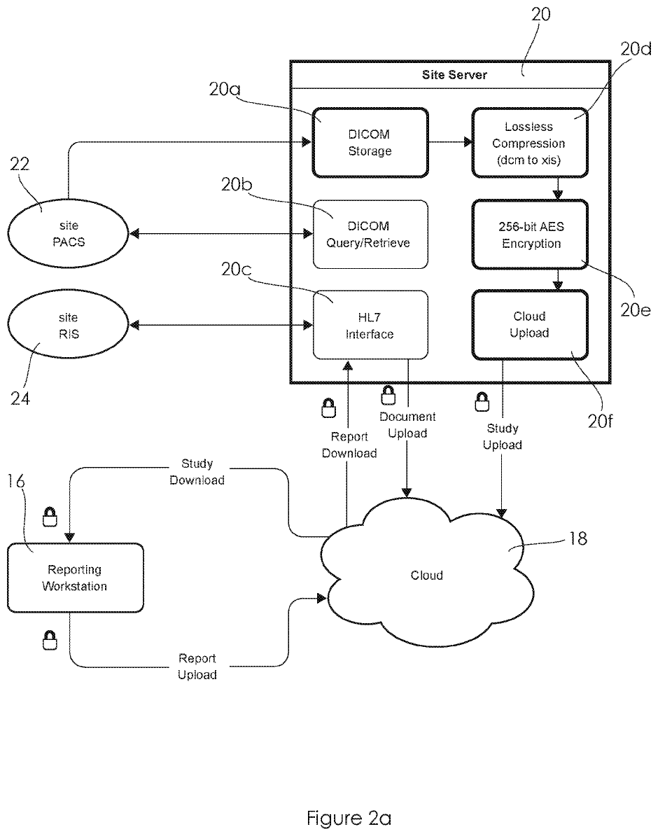

Turning more specifically to the function of the local “site” server 20 and referring to FIGS. 2a and 2b, the local server 20 provides several functions or function modules including DICOM storage 20a, a DICOM Query/Retrieve, a HL7 Interface, Lossless Compression 20d, Encryption 20a and upload/cloud interface 20f.

The system 20, in particular the local server 20, may be configured to operate a method 200 that may include one or more of the following steps, including at step 205, receiving medical image data in the form of DICOM data files from the PACS system 22 into the DICOM storage 20a, and at step 210 performing a DICOM Query/Retrieve 20b function to retrieve prior related imaging data PACS system 22.

At step 215, the local server 20 may be configured to perform file conversions and compression of image data from DICOM files using the lossless compression 20d function, as is further detailed in method 400 below, and at step 220 the converted and compressed files are encrypted by the encryption function 20e and then at step 225, the converted and compressed files are uploaded to the cloud server 18 by the upload function 20e.

At step 230, the local server 20 is configured to retrieve prior reports from RIS system 24 via the HL7 interface 20c, upload of prior reports to cloud server 18, retrieve request documents from RIS system 24, and if required, upload request documents to cloud server 18. At step 235, the local server 20 is configured to retrieve validated reports from cloud server 18 and, at step 240, the local server 20 is configured to communicate validated report to RIS system 24, again using the HL7 interface 20c.

It is noted that the local server 20 is configured to perform all of the functions that require integration with the customers' existing systems within their local network, that may be a VPN (Virtual Private network). It is also noted that the local server 20 is configured to utilised standard protocols to communicate with the PACS system 22 including those for DICOM data such as C-FIND, C-MOVE and C-GET. Communications with the RIS system 24 include use of both the HL7 protocol and vendors custom APIs, as required.

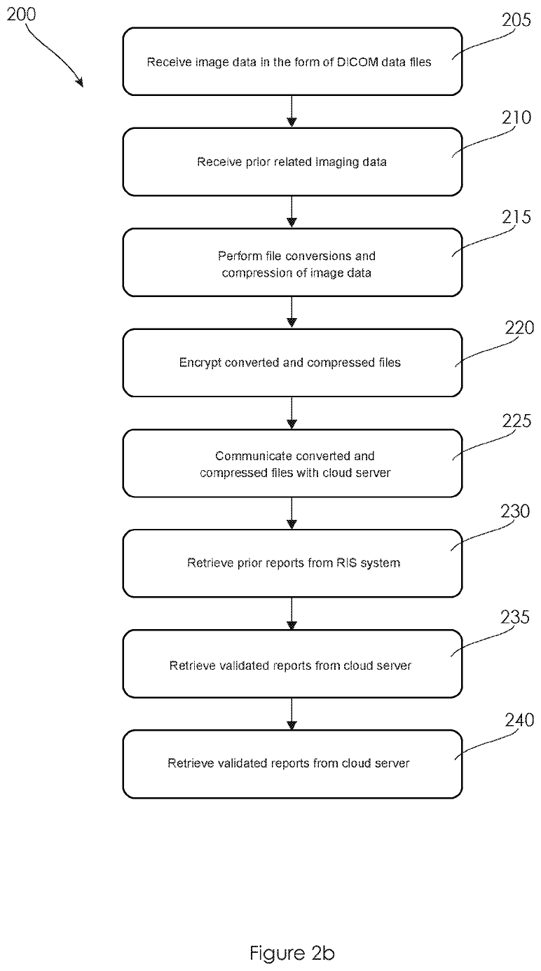

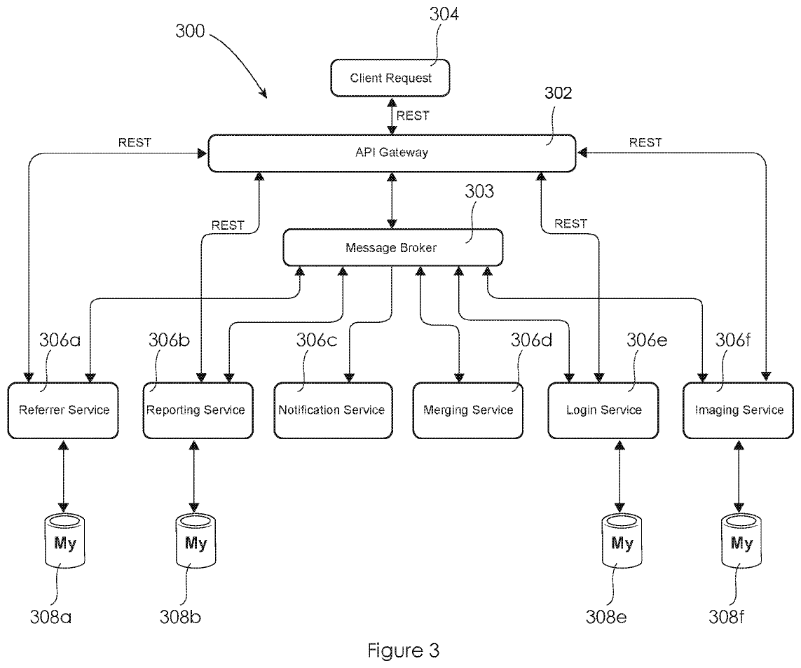

Turning now to the cloud server 18, the cloud server 18 may include a plurality of servers and related computing systems or devices that may provide a cloud service having a microservice architecture 300 as shown in FIG. 3. The microservice architecture 300 includes an API gateway 302 that handles client requests 304 from the local server 20 and/or the one or more client terminals 18.

The API gateway 302 communicates with a plurality of service systems 306 either directly via REST (Representational State Transfer) protocol or via an inter-service message broker 303 as shown in this example. The API gateway 302 functions to validate access tokens and forwards requests to relevant services 306.

The plurality of service systems 306 are configured to provide, but not limited to, a refer service 306a, a reporting service 306b, a notification service 306c, a merging service 306d, a login service 306e and an imaging service 306f The referrer service 306a tracks referrer links and report embedding tasks, the reporting service 306b manages requests, and reports, the notification service 306c provides notifications for client applications based on server events, the merging service 306d merges multiple compressed series files (.xin, .xis) together into a single series file (.xis) if uploaded separately, the login service 306e manages user login credentials and API access tokens and the imaging service 306f accesses compressed images (.xin, .xis) and provides access to contents via web API. Further services may include an update service handles updating the desktop application in the client terminals.

Each of the service systems 306 may have an associated database 308a to 308f. Example tasks performed by the cloud server 18 include: management of imaging data; management of documents; management of worklists; distributing software updates and user management.

Turning now to the image compression subroutine 400, the local server 20 is configured to, at step 405, process the medical imaging study data that includes separating the imaging data from the metadata. In this example, image data may be an image format such as DICOM. The DICOM image format is commonly used to store medical imaging. This format contains both imaging data and a large amount of metadata.

At step 410, the extracted metadata is converted to a JSON (JavaScript Object Notation) format using the instance UID (Unique Identifier) as the filename, and at step 415, the imaging data is extracted to its native file type to provide native image data. This may be accomplished using an existing DICOM library such as dcm4che.

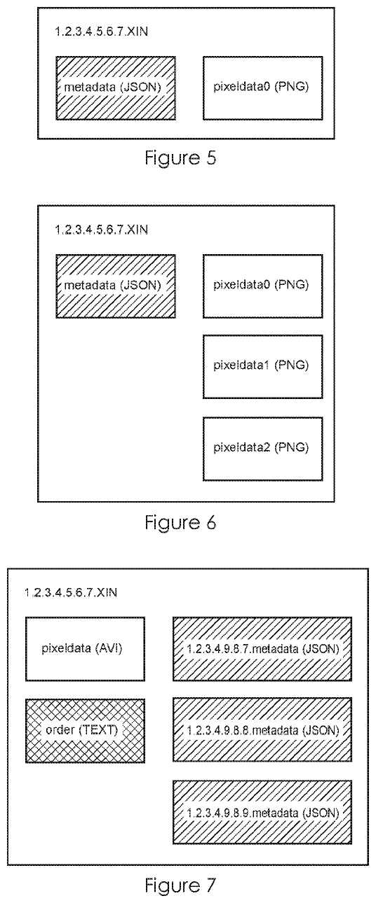

At step 420, the local server 20 is configured to create a first “instance” file type by converting the native image data to PNG format or other suitable lossless image compression format. The images are stored with the filenames “pixeldataN” where N is the frame number starting from 0. The compressed PNG image files and the associated the JSON metadata may be then compressed and packaged within a ZIP file and encrypted using a key assigned for the study. The resulting file format is given a first file type extension. An example of the first file type is shown in FIG. 5 for a single frame instance, and FIG. 6 shows a multi-frame instance.

Once all of the files of the study data are in the first file type, at step 425, a format selection or “compatibility” routine is performed by the local server 20. The format selection routine includes performing an evaluation of the first file types to determine suitability to convert to a second “sequence” file type. The format selection routine including the following selection or compatibility criteria: image dimensions (width and height) for all images must be equal; and each instance must contain a single frame.

If either of these criteria are not satisfied, or a problem is encountered when converting to the second file format, the images are stored using the first file format. It is noted that the second file format is used for volumetric or sequential image data, the first file format is used for all other imaging data.

At step 430, a conversion routine is performed in which compatible compressed images of first file types are converted to create the second file type. The conversion routine includes converting a collection of imaging data from suitable first file types using a lossless video format, such as FFV1, and creating a single “video” file, this may be achieved through a video conversion tool such as FFmpeg. The resulting file is named “pixeldata”.

At step 435, an associated mapping file is created to link the video frames to the associated metadata files. The mapping file contains the instance UID for each of the frames in the order that they appear in the video. Each UID is separated by a line break. The mapping file is given the filename ‘order’.

At step 445, the resulting video file, metadata files, and mapping file are compressed and bundled into a ZIP file encrypted with a key or identifier associated with the study data to which the imaging belongs. The encryption may be, for example, AES 256-bit encryption. However, other suitable encryption methods may be employed.

The resulting file is given the file extension to identify the second file type. An example of the second file format is shown in FIG. 6. The first and second file types may then be communicated with the cloud server 18 and accessed by the one or more client terminals 16.

It is noted that at the one or more client terminals 16, to open the second file type file, the key for the associated study is used to decompress the zip file container. The frames from the video file are extracted to PNG using a tool such as FFmpeg. The extracted frames are matched with their metadata files using the ‘order’ file. These pairs are then used to create a first file type for each instance.

To open the first file type, the key for the associated study is used to decompress the zip file container. The metadata and image data may then be converted back to the DICOM format via a tool such as dcm4che. Within the system 10, the files are used and stored with the metadata in JSON format, and the imaging in the “compressed” PNG format.

Advantageously, the image encryption and compression subroutine 400 allows formatting of the data into first and second file formats that then allows substantially lossless compression and encryption for secure, fast and efficient transfer of the data to the cloud server 18 and ultimately the one or more client terminals. The compressed data also requires lower storage, less bandwidth and thereby may provide cost savings as well as increased speed of data transfer.

Referring now to FIGS. 8a to 17c, there is shown a graphical user interface 500 that may be operated by the desktop application to view the opened first and second file types as outlined above.

The graphical user interface is adapted to provide an intuitive and flexible user interface allowing customization of the layout of medical imaging within the application window. To recap, medical imaging is categorised in a number of steps: a case may contain one or more imaging procedure (such as MRI, CT-Scan etc.); an imaging procedure is classed as a study; each sequence of images within the study is classed as a series; each series contains one or more instance; each instance contains one or more frames.

When a case is opened in the application, one or more studies are loaded. These studies often contain multiple series. It is common for users to view multiple series simultaneously when reporting a case. The focus of the graphical user interface 500 is to provide the user with the tools required to manage and customise the display of these series within the application window. The graphical user interface 500 may be configurable between a number of views of a window of a browser or screen, as are further detailed below.

Referring firstly to FIG. 8a, the graphical user interface 500 may be configurable to show include one or more windows or layouts 501 that display one or more series viewers 505 and a selection region 503 provided here in the form of a series selection bar 504. Accordingly, multiple series viewers may be displayed simultaneously. Each series viewer 505 includes a number of controls 506 in the corners of the viewer allowing the user to access various tools and displays.

The configuration of the layout 501 of the graphical user interface 500, may use icons that illustrate the selectable screens and views to the user. In this example, there are two types icon types provided being a series icon 510 and a viewer icon 515. The series icons 510 represents an imaging series and the viewer icon 515 represents a viewer displaying the imaging series.

The series icons 510 are shown in the series selection bar 504 and indicate the selection of series icons 510 that are selected to add to the window 501. In this example, four example series viewers 505 are shown and each series viewer 505 has a viewer icon 515 displayed, in this example in the bottom left hand side of the series viewer 505.

Referring in addition FIG. 8b, an example of a single series viewer 505 is shown which is a portion of the application window as shown in FIG. 8a that displays imaging from a single series and in FIGS. 9a and 9b details views of icon types are provided being the series icon 510 shown in an unselected state and a selected state, 510a and 510b, in FIGS. 9a and 9b respectively, and the viewer icon 515 as shown in FIG. 10.

The dragging of the icons is implemented using the standard drag and drop features provided by the browser or operating system. The graphic used for the drag operation is the icon itself.

In more detail, the series icon 510, as shown in FIGS. 9a and 9b, is used to represent a single imaging series. It includes a thumbnail 511 displaying an image from the series, and indicia 512 that may include a description 512a of the series in the area below the thumbnail, and a number 512b overlayed in the top right corner of the thumbnail indicating the number of frames within the series. Series icons 510 are used wherever series are listed. The user may drag and drop the series icon 510 to dynamically add a viewer displaying the dragged series.

The viewer icon 515 is used to represent a viewer displaying an imaging series. The view icon 515, as shown in FIG. 10, is displayed at the bottom left of the related series viewer (see for example FIGS. 8a and 8b) and includes a thumbnail image 516 of the displayed series inside of an indicia 517 in the form of a circle. The user may drag and drop the viewer icon 515 similarly to the series icon 510.

When a series or viewer icon is dragged, there are a number of drop zones available on both the application window and the viewer sections.

### Window Drop Zones

The window drop zones are located at a plurality of locations around the edge of the application window that allow the user to dynamically add an additional series viewer. The drop zones cover about, but not limited to, about an outer 5% of the window frame. These drop zones are shown in FIG. 11 is ID [1, 2, 3 & 4], with the action performed by dropping the icon within each drop zones described in Table 1 below.

TABLE 1
Drop Zone Functionality

| Zone | | | |
|---|---|---|---|
| ID | Action | Width | Height |
| 1 | Add a new series viewer to theleft of the window. | 5% | 100% |
| 2 | Add a new series viewer to the top of the window. | 90% | 5% |
| 3 | Add a new genes viewer to the right of the window | 5% | 100% |
| 4 | Add a new series viewer to the bottom of the | 90% | 5% |
| | window. | | |

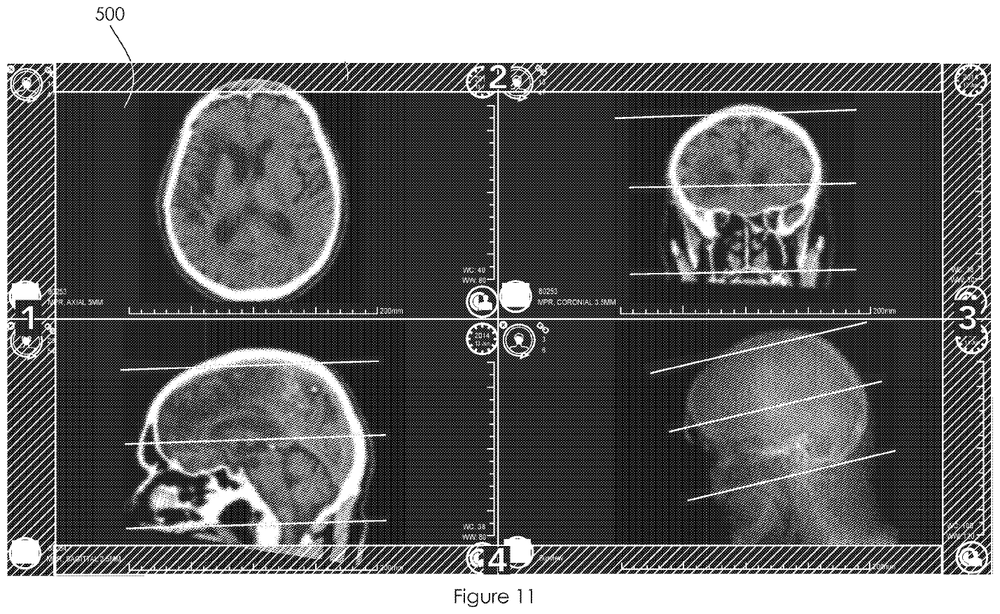

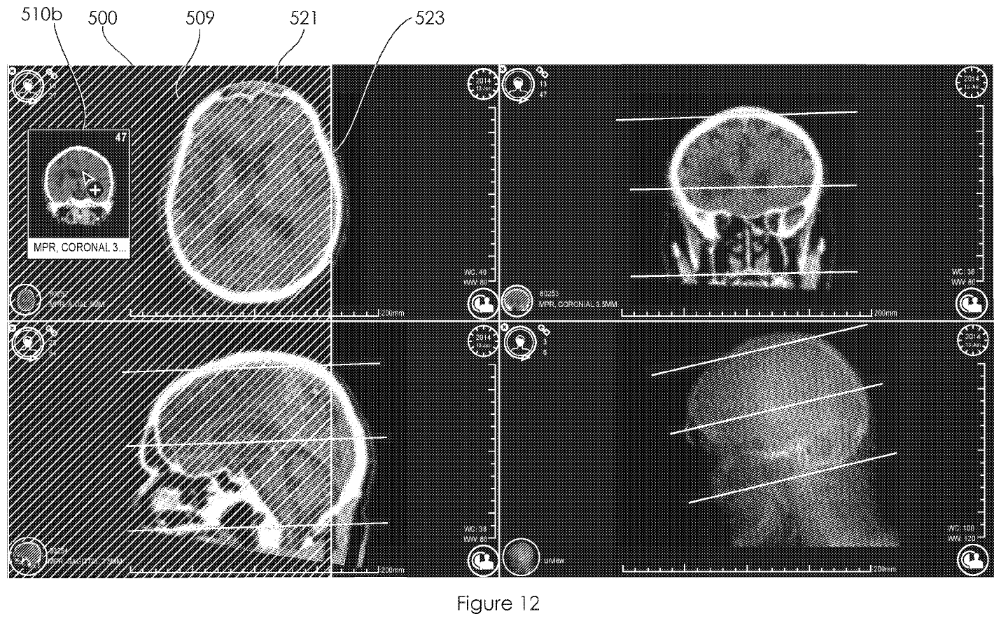

When an icon is dragged over one of these drop zones, a drop indicator 509 is displayed showing the user the location at which the new series viewer will be located. This drop indicator may include a drop indicator indicia 512 provided here in the form of a translucent grey box cross-hatch shading that has an edge feature 523 such as, for example, a coloured green line along the edge that will border the existing viewers. An example of this drop indicator 509 can be seen in FIG. 12.

### Viewer Drop Zones

In the case that a window contains no existing series viewers, an additional drag option is available that will add a series viewer filling the window. The drop zone for this operation covers the entire window and the drop indicator is a translucent grey box, shown here in crosshatch, filling the window.

When dragging over an existing series viewer, a number of additional drop zones are available that allow the existing series viewer to be split, replaced, or swapped. The splitting function is available for both the series icon and viewer icons, however the replace action is limited to series icons, while the swap action is limited to viewer icons.

The split drop zones cover the outer 25% of the series viewer frame, while the replace/swap drop zone covers the middle 20% of the series viewer frame. The drop zones are shown in FIG. 13, while the actions for each zone are described in Table 2 below.

TABLE 2
Series Viewer Drop Zone Actions

| Zone | | | |
|---|---|---|---|
| ID | Action | Width | Height |
| 1 | Split the existing series viewer with the new series | 25% | 100% |
| | viewer to the left and the existing series viewer to | | |
| | the right. | | |
| 2 | Split the existing series viewer with the new series | 50% | 25% |
| | to the top and the existing series viewer to the | | |
| | bottom. | | |
| 3 | Split the existing seies viewer with the new series | 25% | 100% |
| | viewer to the right and the existing series viewer | | |
| | to the left. | | |
| 4 | Split the existing series viewer with the new series | 50% | 25% |
| | to the bottom and the existing series to the top. | | |
| 5a | When a series icon is dragged, the existing series | 20% | 20% |
| | viewer is replaced entirely with one displaying the | | |
| | dragged series. | | |
| 5b | When a viewer icon is dragged, the source and | 20% | 20% |
| | destination series viewers swap locations. | | |

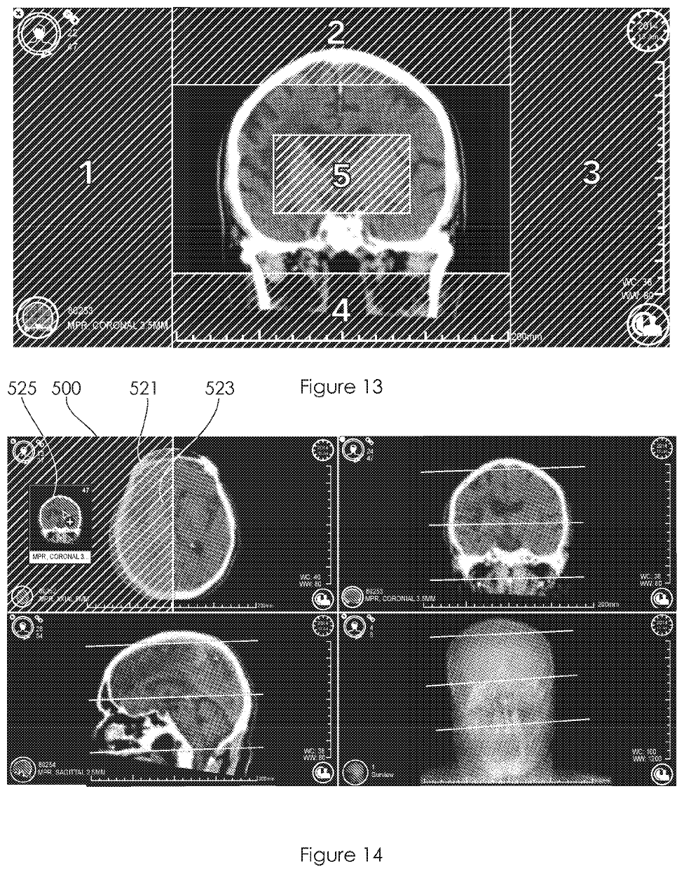

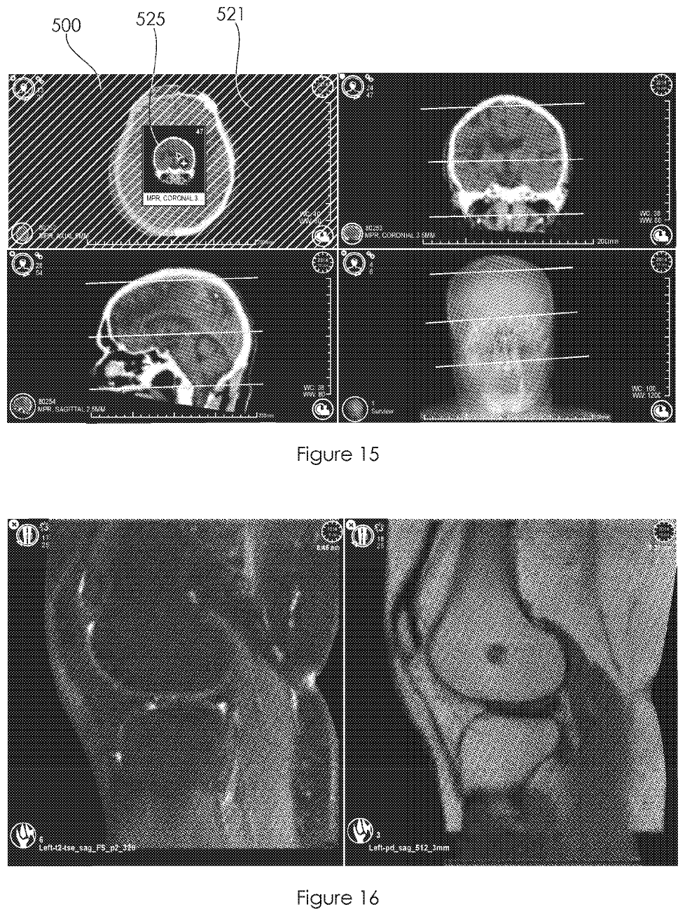

When a series or viewer icon are dragged over one of these drop zones, a preview 525 of the location of the new series viewer is displayed, as can be seen in FIG. 14 and FIG. 15. The indicia 521 in the form of the translucent/cross-hatched grey box is displayed with the edge feature 523 that may be now a red edge along the join of the split viewers in the case of a split drop zone. In the case of a replace/swap drop zone the translucent/cross-hatched grey box is displayed with a red border inset from the edge of the existing series viewer. Other edge features or indicators other than red or green boarders may be used such as bold lines or markers.

### Overlay Drag

By pressing a modifier key while dragging, the user may overlay one series on another. A preview of the overlay series is displayed if the two series are compatible, and the overlay permanently applied when the drop is completed. Two series are considered compatible if they share a common reference frame, as shown in FIG. 16.

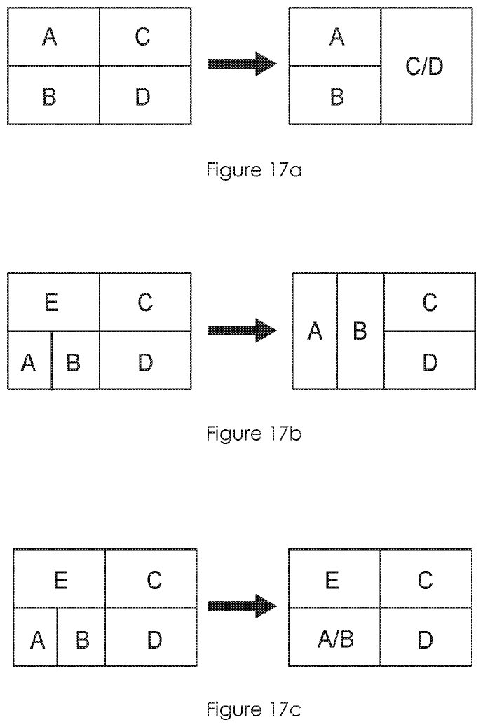

A series viewer may be closed via the “x” icon in the top left-hand corner of each viewer. When closing the viewer, the layout is updated to fill the space vacated by the closed viewer. The space is filled in a way that causes the minimum amount of change from the previous layout; favouring a vertical fill; examples outlining some fills are shown in FIGS. 17a, 17b and 17c.

In summary, there had been described a system, methods and an interface that seek to improved multiple aspects of teleradiology. The configuration of the system, in particular, use of the local server, enables communication with the cloud server without using the VPN which simplifies configuration, allows greater bandwidth and hence speed. The pre-caching (or pre-downloading) by the application of the remote client terminals avoids problems with streaming and improves speed and usability at the client terminal. The system allows use of a single application that access the services at the local system via a secure public URL, thus not requiring the VPN.

Most advantageously, the image encryption and compression method allow formatting of the data into first and second file formats that then allows substantially lossless compression and encryption for secure, fast and efficient transfer of the data to the cloud server and ultimately the one or more client terminals. The compressed data also requires lower storage and thereby may provide cost savings.

Further advantageously, the graphical user interface seeks to provide intuitive and flexible user interface allowing customization of the layout of medical imaging within the application window. The use of images as the icons that may be dragged and dropped into drop zone to provide further views and splits reduces the number of operations a user needs to make to obtain the overall desired view or set of views.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any known matter or any prior publication is not, and should not be taken to be, an acknowledgment or admission or suggestion that the known matter or prior art publication forms part of the common general knowledge in the field to which this specification relates.

While specific examples of the invention have been described, it will be understood that the invention extends to alternative combinations of the features disclosed or evident from the disclosure provided herein.

Many and various modifications will be apparent to those skilled in the art without departing from the scope of the invention disclosed or evident from the disclosure provided herein.

1.-21. (canceled)

22. A method for processing medical imaging study data for communication between a local system and a client terminal, the method including the steps of:
a. Processing, at the local system, the study data into image data and associated meta data;
b. Compressing, at the local system, the image data;
c. Creating, at the local system, a first file type including the compressed image data and associated meta data;
d. Processing, at the local system, the compressed image data associated with a plurality of the first file types to determine compatible compressed images based on a compatibility criteria;
e. Creating, at the local system, a second file type by combining a plurality of the compatible compressed images into a video format;
f. At the local system, at least one of encrypting and further compressing one or more of the first file type and the second file type suitable for communication with the client terminal.

23. The method according to claim 22, wherein the step of creating the second file type further includes:
a. Creating a mapping file to link video frames of the video format to the associated metadata files.

24. The method according to claim 22, wherein the compatibility criteria includes:
a. Image dimensions of the compressed image data must be equal; and
b. An instance of medical imaging study data must contain a single frame.

25. The method according to claim 22, wherein the image data is compressed using a lossless compression format to provide the compressed image data.

26. The method according to claim 25, wherein the lossless compression format is PNG.

27. The method according to claim 25, wherein the plurality of the compatible compressed images are combined with lossless video compression format to provide the video format.

28. The method according to claim 27, wherein the lossless video compression format is FFmpeg.

29. The method according to claim 22, wherein the local system is within a virtual private network.

30. A system for processing medical imaging study data for communication between a local system and a client terminal, the system including a local system and a remote client terminal, the system being configurable to:
a. Process, at the local system, the study data to into separate image data and associated meta data;
b. Compress, at the local system, the image data;
c. Create, at the local system, a first file type including the compressed image data and associated meta data;
d. Process, at the local system, the compressed image data associated with a plurality of the first file types to determine compatible compressed images based on a compatibility criteria;
e. Create, at the local system, a second file type by combining a plurality of the compatible compressed images into a video format;
f. At the local system, at least one of encrypt and further compress one or more of the first file type and the second file type suitable for communication with the client terminal.

31. A configurable interface for display of medical image data, the interface being shown on a window of a computer display, the interface including:
a. one or more series icons that each represent a single imaging series;
b. one or more series viewers that are each locatable in a layout in at least a portion of the window to display the single imaging series associated with each of the respective one or more the series icons; and
c. one or more viewer icons that are each associated with a respective one or more of the series viewers,
d. wherein each of the one or more series icons and one or more viewer icons are draggable over one or more of a plurality of drop zones associated with the at least one of the window and the one or more of the series viewers, each of the plurality of drop zones having different locations within the respective at least one of the window and one or more series viewers and each having different layout modification functions associated with each of the different locations,
e. wherein the dragging of the one or more series icons and one or more viewer icons over each of the different locations of one of the plurality of drop zones provides a dynamic indication of a modified layout to be applied by the different layout modification functions associated with the different locations, and then introduces the modified layout to be displayed on the window upon dropping of the dragged one or more series icons and one or more viewer icons.

32. The configurable interface according to claim 31, wherein the indication of the modified layout includes an indicator displayable over at least one of the window and one or more series viewers.

33. The configurable interface according to claim 32, wherein the indicator provides a preview of the modified layout.

34. The configurable interface according to claim 33, wherein the preview is at least one of a translucent or cross-hatched box.

35. The configurable interface according to claim 32, wherein the indicator includes at least one of a colour, a marking, and an edge feature associated with the one or more series viewers.

36. The configurable interface according to claim 31, wherein each of the one or more series icons includes thumbnail image representative of an image associated with the single imaging series and an associated indicia to indicate associated image information.

37. The configurable interface according to claim 31, wherein each of the one or more viewer icons includes thumbnail image representative of an image associated with single imaging series being displayed and an associated indicia to indicate associated image information.

38. The configurable interface according to claim 31, wherein each of the one or more series icons is displayable over the associated series viewer.

39. The configurable interface according to claim 31, wherein the plurality of drop zones include a plurality of window drop zones adapted to allow the one or more series icons to be dropped thereat to dynamically add an associated one or more series viewers to the window.

40. The configurable interface according to claim 39, wherein the plurality window drop zones are located toward and around an edge of the window.

41. The configurable interface according to claim 31, wherein the plurality of drop zones include a plurality of viewer drop zones.
